(19) United States
(12) Patent Application Publication
(10) Pub. No.: US 2013/0323217 A1
(43) Pub. Date: Dec. 5, 2013

(54) COMPOSITIONS AND METHODS FOR THE CONTROLLED REPOPULATION OF GRAM-NEGATIVE BACTERIA IN THE COLON

(71) Applicants: Antoine Andremont, Malakoff (FR); Jean De Gunzburg, London (GB)

(72) Inventors: Antoine Andremont, Malakoff (FR); Jean De Gunzburg, London (GB)

(73) Assignees: Da Volterra, Paris (FR); ASSISTANCE PUBLIQUE - HOPITAUX DE PARIS, Paris (FR); Universite Paris Diderot-Paris 7, Paris (FR)

(21) Appl. No.: 13/991,013

(22) PCT Filed: Dec. 22, 2011
(86) PCT No.: PCT/EP2011/073866

§ 371 Date: Aug. 12, 2013

(30) Foreign Application Priority Data
Dec. 22, 2010 (EP) 10306492.9

(51) Int. Cl. A61K 35/74; A61K 38/12; A61K 45/06; A61K 31/7036

(52) U.S. Cl. CPC A61K 35/741; A61K 31/7036; A61K 38/12; A61K 45/06
USPC 424/93.48

(57) ABSTRACT

The present invention relates to a method for the controlled repopulation of the colonic bacterial flora.

The present invention relates to a method for the controlled repopulation of the colon of a subject who has undergone selective digestive decontamination.

Since their discovery, antibiotics have substantially reduced the threat posed by infectious diseases. However, these gains are now jeopardized by the emergence and spread of microbes that are resistant to antibiotics. The consequences are severe. Infections caused by resistant microbes fail to respond to treatment, resulting in prolonged illness and greater risk of death. Treatment failures also lead to longer periods of infectivity, which increase the number of infected people moving in the community and thus expose the general population to the risk of contracting an infection caused by a resistant strain. Bacteria are particularly efficient at amplifying the effects of resistance, not only because of their ability to multiply very rapidly but also because they can transfer their resistance genes that are often carried by mobile genetic elements such as plasmids. Resistance to a single drug can thus spread rapidly through a bacterial population. When anti-microbials are used incorrectly (which is regrettably extremely frequent), the likelihood that bacteria and other microbes will adapt (by becoming resistant) and multiply rather than be killed is greatly enhanced.

A further threat is presented by the fact that bacteria are now often simultaneously resistant to several antibiotics from different classes; this stems from the linkage of several genes conferring resistance to different antibiotics on the same mobile genetic element (up to 9 such genes have been found on the same plasmid). Hence, the treatment of a patient with a single antibiotic may select for the resistance to multiple antibiotics, including to drugs that would normally be reserved for the treatment of serious cases in hospital settings. This is why clinicians often refer to multidrug-resistant bacteria.

As the number of infections and the corresponding use of antibiotics have increased, so has the prevalence of resistance. In addition, the enhanced food requirements of an expanding world population have led to the widespread routine use of antibiotics as growth promoters or preventive agents in food-producing animals and poultry flocks. Such practices have likewise contributed to the rise in resistant microbes, which can be transmitted from animals to man. Hospitals are also a critical component of the antibiotic resistance problem worldwide. The combination of highly susceptible patients, intensive and prolonged antibiotic use, and cross-infection have resulted in nosocomial infections with highly resistant bacterial pathogens, often resistant to multiple classes of antibiotics. Resistant hospital-acquired infections are expensive to control and extremely difficult to eradicate. Hospitals are also the last resort site of treatment for many patients with severe infections due to resistant pathogens acquired in the community.

In a number of instances the colon is colonized by antibiotic-resistant and potentially pathogenic bacteria, such as Enterobacteria or other Gram-negative bacteria such as Pseudomonas, Acinetobacter or other non fermentative Gram-negative bacteria. This occurs particularly when the intestinal colonic microbiota has been disrupted. Disruption of the intestinal microbiota can result from different causes, but the predominant one is the previous intake of antibiotics, the residues of which often reach the colon under active form. (Donskey C J. Antibiotic regimens and intestinal colonization with antibiotic-resistant Gram-negative bacilli. Clin Infect Dis. 2006 Sep. 1; 43 Suppl 2:S62-9. Review.)

Indeed, the intestinal colonic microbiota is a complex ecosystem comprising several hundreds of bacterial species, some of them being responsible for eliminating exogenous micro-organisms, including Gram-negative bacteria which are multiresistant to antibiotics and/or potentially pathogenic for the host. This capacity of the intestinal microbiota to eliminate exogenous microorganisms is called “colonisation resistance” (van der Waaij D, Berghuis J M, Lekkerkerk J E. Colonization resistance of the digestive tract of mice during systemic antibiotic treatment. J Hyg (Lond). 1972 December; 70(4):605-10.)

Orally administered antibiotics have been used to eliminate resistant and/or potentially pathogenic Gram-negative bacteria from the gut of individuals carrying such bacteria amongst their colonic commensal flora. This practice is referred to “selective digestive decontamination” or SDD (Selective Decontamination of the Digestive tract. Smet A M, Bonten M J. Curr Opin Infect Dis. 2008 April; 21(2):179-83). It aims at eliminating, or strongly reducing, the intestinal colonisation and subsequent spread of commensal and/or potentially pathogenic bacteria, such as Gram-negative bacteria resistant to most, or all beta-lactam antibiotics, as well as sometimes antibiotics from other classes, the resistance to antibiotics of which can cause medical or public health problems. This is in particular applied to very sick patients at high risk of infection (such as intensive care, or haemato-oncology patients, for example) before they actually develop a real infection.

The present inventors have already proposed innovative selective decontamination with site-specific release of antibiotics in the colon (see applications WO2009/037264 and WO2010/103119).

While selective decontamination can be effective, it is never completely selective for the target intestinal bacteria and, most of the time, part of the normal microbiota is also affected and resistance to colonisation affected. The inventors have observed that in many cases, a wave of recolonization of the colon with resistant bacteria can occur shortly after the end of the administration of the decontamination regimen because, at that time, the normal bacterial flora which is responsible for colonisation resistance is not yet restored. It would thus be advantageous to have at hand a method for the controlled repopulation of the commensal intestinal flora after elimination of antibiotic-resistant Gram-negative bacteria from the colon.

Compositions and methods for providing controlled repopulation (or otherwise referred to as reconstruction) of the commensal flora of the colon in a subject who has undergone selective digestive decontamination are thus disclosed.

The invention stems from the discovery that after decontamination of the colon with anti-Gram-negative antibiotics, recolonization can be achieved in a controlled manner by administering to a subject in need thereof a non-pathogenic, non-antibiotic resistant E. coli strain, for example E. coli strain Nissle 1917 (DSM 6601). The compositions and methods according to the invention can, therefore, be used to reduce or eliminate the above-mentioned drawbacks related to the use of antibiotics.

The present invention relates in particular to a method for the repopulation of the commensal flora of the colon of a subject in need thereof who has been previously subjected to a Gram-negative bacteria selective decontamination of its colon, comprising administering to the subject a composition comprising at least one non-pathogenic, non-antibiotic-resistant E. coli strain. In a preferred embodiment, said strain is E. coli strain Nissle 1917 (DSM 6601).

A further object of the present invention relates to a non-pathogenic, non-antibiotic-resistant E. coli strain, in particular E. coli strain Nissle 1917, for use in a method for the repopulation of the commensal flora of the colon of a subject in need thereof who has been previously subjected to a Gram-negative bacteria selective decontamination of its colon.

Another object of the invention relates to the use of a non-pathogenic, non-antibiotic-resistant E. coli strain, in particular E. coli strain Nissle 1917, for the manufacture of a medicament intended to repopulate the commensal flora of the colon of a subject in need thereof who has been previously subjected to a Gram-negative bacteria selective decontamination of its colon.

In the context of the present invention, a decontamination that is selective for the targeted antibiotic-resistant Gram-negative bacteria is preferable as it is sought to limit the impact of the regimen on the flora. A subtle equilibrium is present in the normal commensal flora and the decontamination that is described herein is aiming to preserve the normal functions of the flora while eliminating the resistant bacteria that are seen as the cause of subsequent spread of commensal and/or potentially pathogenic bacteria. The decontamination protocol should aim to spare the commensal bacteria that are not the cause of concern. Consequently, the selective digestive decontamination described in the invention rules out intestinal lavages and other techniques aiming to fully remove the intestinal flora such as administration of antibiotics having a broad-spectrum activity against Gram-positive and Gram-negative bacteria, like metronidazole, fosfomycin or others. The decontamination of the invention is not a complete or near-complete elimination of the flora. The compositions and methods of the invention should aim to preserve (by the selectivity of the decontamination), and restore (by providing controlled repopulation) the beneficial resistance to colonisation of the flora. Accordingly, Gram-negative bacteria decontamination is provided by a treatment with one or several means recognized for their ability to eliminate preferentially Gram-negative bacteria as compared to Gram-positive bacteria. As mentioned below, use of an antibiotic selective for Gram-negative bacteria antibiotics is particularly preferred. As such, in the context of the present invention “anti-Gram-negative antibiotic” denotes an antibiotic which is essentially selective for Gram-negative bacteria as compared to Gram-positive bacteria. In addition, given the aim of the present invention, which is to reduce or eliminate antibiotic-resistant bacteria in the colon of a subject, the selective digestive decontamination implemented herein does preferably not comprise the classical SDD initial step of parenteral administration of broad-spectrum antibiotics to treat infections incubating at the time of admission of the subject in a care unit.

The Escherichia coli strain Nissle 1917 (DSM 6601 in the German Collection for Microorganisms) is one of the best-characterised and therapeutically relevant bacterial strains worldwide. E. coli strain Nissle 1917 has no identified pathogenic characteristics and does not carry antibiotic-resistance genes. This E. coli has been shown to be able to colonize the intestine of piglets (Barth et al., Journal of applied microbiology, 107 (2009) 1697-1710) and is effective in inhibiting the adhesion and invasion of intestinal epithelial cells by adherent-invasive E. coli strains (Altenhoefer et al., FEMS Immunol Med Microbiol. 2004, 40(3); 223-229; Boudeau et al., Aliment Pharmacol Ther. 2003; 18(1), 45-56). However, before the present invention, it has never been shown that non-pathogenic, non-antibiotic-resistant E. coli strains such as E. coli strain Nissle 1917 can be used to prevent the repopulation of the colon with resistant bacteria after selective decontamination. The present inventors have shown that the faeces of piglets that have undergone selective decontamination with colistin present a reduced repopulation with 3^{rd} generation cephalosporin-resistant bacteria when administration of such a non-antibiotic-resistant E. coli strain (e.g. E. coli strain Nissle 1917) is administered after decontamination.

The present invention implements administering to the subject any non-pathogenic, non-antibiotic-resistant E. coli strain which has advantages similar to those mentioned above for E. coli strain Nissle 1917 (no identified pathogenic characteristics and does not carry antibiotic-resistance genes). Representative alternative strains include among others E. coli strain M17 (e.g., the ProBactrix product sold by BioBalance) and E. coli strain GUT-DSM 16481 (e.g., the Rephalysin product sold by Repha Gmbh). Symbioflor 2 available from ENERGETIC NATURA is also a product containing an alternative non-pathogenic, non-antibiotic resistant E. coli strain that can be used according to the invention.

The non-pathogenic, non-antibiotic resistant E. coli strain can be administered in different ways. In a particular embodiment, the E. coli strain is administered orally. In a specific form of the invention, the non-pathogenic, non-antibiotic resistant E. coli strain is administered in the form of an oral gastro-resistant composition. In a further particular embodiment, the non-pathogenic, non-antibiotic resistant E. coli strain is formulated such as to be orally administrable and released in the distal part of the gastrointestinal tract, in particular in the ileum, the caecum or the colon.

The present invention further relates to a method for the elimination of antibiotic-resistant Gram-negative bacteria in the colon of a subject in need thereof, comprising:

a) the elimination of Gram-negative bacteria, including resistant strains, from the colon;
b) the repopulation of the colon of the subject with a non-pathogenic, non-antibiotic-resistant E. coli strain.

An object of the invention is therefore related to a sequential treatment of the subject, comprising the selective suppression of Gram-negative bacteria in the colon and a restoration phase to reconstruct and enhance the susceptible flora in a sustained way.

Another object of the invention relates to a method for reducing or eliminating the dissemination of Gram-negative resistant strains in the environment, and in particular in hospital settings, comprising treating a subject in need thereof by:

Step a) eliminating Gram-negative bacteria, including resistant strains, from the colon of said subject;
Step b) repopulating the colon of the subject with a non-pathogenic, non-antibiotic-resistant E. coli strain.

The method thus involves the selective elimination of Gram-negative bacteria from the colon and its subsequent repopulation with a non-pathogenic, non-antibiotic-resistant E. coli strain.

The subject may be an animal, in particular a mammal, more particularly a human. The subject is a carrier of antibiotic-resistant Gram-negative bacteria in the colon, but is not necessarily in a disease state. The subject may in particular be a person whose colon comprises antibiotic-resistant Gram-negative bacteria, but not necessarily pathogenic bacteria. It can in particular be a patient whose faeces samples have been analyzed and shown presence of antibiotic-resistant Gram-negative bacteria, but who is not ill. The subject may also be a patient suffering from a disease originating from antibiotic-resistant Gram-negative bacteria, or from any other origin.

The invention could also be applied to patients with less severe conditions, such as subjects returning from travel abroad because they are at increased risk to be colonised by multiresistant Gram-negative bacteria and at higher risk of infections by these bacteria (Freeman J T, McBride S J, Heffernan H, Bathgate T, Pope C, Ellis-Pegler R B. Community-onset genitourinary tract infection due to CTX-M-15-Producing Escherichia coli among travelers to the Indian subcontinent in New Zealand. Clin Infect Dis. 2008 Sep. 1; 47(5):689-92.; Kumarasamy K K, Toleman M A, Walsh T R, Bagaria J, Butt F, Balakrishnan R, Chaudhary U, Doumith M, Giske C G, Irfan S, Krishnan P, Kumar A V, Maharjan S, Mushtaq S, Noorie T, Paterson D L, Pearson A, Perry C, Pike R, Rao B, Ray U, Sarma J B, Sharma M, Sheridan E, Thirunarayan M A, Turton J, Upadhyay S, Warner M, Welfare W, Livermore D M, Woodford N. Emergence of a new antibiotic resistance mechanism in India, Pakistan, and the UK: a molecular, biological, and epidemiological study. Lancet Infect Dis. 2010 September; 10(9):597-602. Epub 2010 Aug. 10.)

It could also be applied to prevent dissemination of intestinal resistant Gram-negative bacteria within households (Tandé D, Boisramé-Gastrin S, Münck M R, Héry-Arnaud G, Gouriou S, Jallot N, Nordmann P, Naas T. Intrafamilial transmission of extended-spectrum-beta-lactamase-producing Escherichia coli and Salmonella enterica Babelsberg among the families of internationally adopted children. J Antimicrob Chemother. 2010 May; 65(5):859-65.)

Step a) described above can be achieved by any means known to those skilled in the art. In a particular embodiment, step a) comprises orally administering to the subject at least one antibiotic selective against Gram-negative bacteria. The antibiotic used for the purpose of eliminating such bacteria should be efficient in that it is able to eliminate the antibiotic-resistant Gram-negative bacteria present in the colon of the subject to be treated. In other terms, the antibiotic-resistant Gram-negative bacteria to be eliminated are not resistant to the antibiotic used in the method of the present invention. The antibiotics which can be used may be identified, for example, by testing resistance among target bacteria in faeces samples from the subject to be treated, as is well known by the person skilled in the art.

In addition, antibiotics which select only few resistant bacteria can be used. Furthermore, a combination of at least two antibiotics can prevent the emergence of resistant bacteria by selection of mutants resistant to any one of either of the components. Indeed, the association of antibiotics, especially if given at high doses, is an effective means of preventing the emergence of such mutants.

Thus, step a) can comprise the administration of at least two antibiotics effective against Gram-negative bacteria. In a specific embodiment, one or more of the at least two antibiotics is formulated to obtain a site-specific delivery. For example, the antibiotics can be formulated to be orally administered compositions that deliver the antibiotics to a part of the intestine selected from the ileum, the caecum or the colon, but not upstream from the ileum.

Suitable Gram-negative selective antibiotics, for use in step a) include, for example, peptide antibiotics, and in particular lipopeptide antibiotics such as polymixins, and in particular colistin (polymixin E), and also aminoglycoside antibiotics can be used.

An aminoglycoside is a molecule composed of a sugar group and an amino group. Several aminoglycosides are effective against Gram-negative bacteria, including amikacin, arbekacin, gentamicin, kanamycin, neomycin, netilmicin, paromomycin, rhodostreptomycin, streptomycin, tobramycin, spectinomycin and apramycin. Particularly preferred aminoglycosides in the invention include neomycin, streptomycin, tobramycin, spectinomycin and gentamicin.

Anti-Gram-negative peptide antibacterials are well-known (See, for example, Hancock et al. Adv. Microb. Physiol. 37:135-175; Kleinkauf et al., 1988. Crit. Rev. Biotechnol. 8:1-32; and Perlman and Bodansky. 1971. Ann. Rev. Biochem. 40:449-464), and fall into two classes, non-ribosomally synthesized peptides, such as gramicidins, polymyxins, bacitracins, glycopeptides, etc., and ribosomally synthesized (natural) peptides. Representative antibacterials that are used commercially include colistin (also known as colimycin or polymyxin E), bacitracin, gramicidin S, and polymyxin B.

In a particular embodiment, step a) comprises administration of an anti-Gram-negative bacteria lipopeptide antibiotic. In a variant of this embodiment, the lipopeptide antibiotic is a polymyxin. As used herein, a “polymyxin” is defined as an anti Gram-negative lipopeptide which is a polycationic decapeptide containing a heptapeptide ring and a fatty acid chain in the N-terminal position. In addition to the lipopeptides, analogs thereof which no longer include the lipid moiety, but which retain anti-Gram-negative efficacy, can be used. A particularly preferred polymyxin used in the present invention is colistin (also known as polymyxin E).

The peptide antibiotic can be orally administrable and formulated such as to be delivered in a site-specific manner. In particular, the peptide antibiotic can be formulated to be delivered in a part of the intestine of the subject selected from the ileum, the caecum or the colon, but not before the ileum.

In another particular embodiment, step a) comprises administration of an aminoglycoside to the subject, either alone or in combination with a peptide antibiotic such as colistin. Representative aminoglycosides include, but are not limited to, neomycin, amikacin, arbekacin, gentamicin, kanamycin, netilmicin, paromomycin, rhodostreptomycin, streptomycin, tobramycin, spectinomycin and apramycin. In a preferred embodiment, the aminoglycoside is neomycin. In a particular embodiment, the aminoglycoside, in particular one of those specific aminoglycosides mentioned above, is orally administrable and formulated such as to be delivered to a specific part of the intestine of the subject. For example, said part of the intestine can be the ileum, the caecum or the colon. Preferably, the aminoglycoside is not delivered upstream from the ileum.

In a preferred embodiment, step a) comprises the administration of colistin and neomycin to the subject. In particular, as mentioned above, colistin can be formulated such as to be delivered to a part of the intestine of the subject selected from the ileum, the caecum or the colon, but not upstream from the ileum. In another particular embodiment, neomycin is or is not formulated such as to be delivered to a part of the intestine of the subject selected from the ileum, the caecum or the colon, but not upstream from the ileum. In a variant, both colistin and neomycin are formulated such as to be delivered to a part of the intestine of the subject selected from the ileum, the caecum or the colon, but not before the ileum, either both in the same formulation, or in separate formulations.

In a particular embodiment, step a) is implemented by using the drug delivery systems for selective decontamination of the colon described in international applications WO2009/037264 and WO2010/103119, which are hereby incorporated by reference.

Step b) comprises the administration of a non-pathogenic, non-antibiotic-resistant E. coli strain, as defined above (e.g. E. coli Nissle 1917, E. coli M17 or E. coli GUT-DSM 16481 strain, among others). A preferred embodiment implements the administration of E. coli strain Nissle 1917 strain (accession number DSM 6601). As mentioned above, said E. coli strain can be formulated as an oral gastro-resistant composition and/or as an oral administrable composition allowing release of the strain in the terminal part of the gastro-intestinal tract, in particular in the ileum, the caecum or the colon.

The person skilled in the art knows different sources of E. coli strain Nissle 1917. In particular, this strain is commercialized by Ardeypharm Gmbh under the name Mutaflor®.

In a particular aspect, the invention relates to a non-pathogenic, non-antibiotic-resistant E. coli strain as defined above, in particular to the E. coli strain Nissle 1917 strain, for use in a method for the repopulation of the commensal flora of the colon of a subject in need thereof who has been previously subjected to a treatment with at least one anti-Gram-negative bacteria antibiotic, and in particular with at least two anti-Gram-negative bacteria antibiotics.

The present invention further relates to a kit comprising at least:
a first composition comprising at least one antibiotic efficient against Gram-negative bacteria, and
a second composition comprising a non-pathogenic, non-antibiotic-resistant E. coli strain, in particular E. coli strain Nissle 1917.

The compositions of the kit can be used in the methods of the present invention, the first composition being used for step a) (decontamination step) and the second composition for step b) (repopulation step).

A particular kit according to the invention comprises a first composition comprising colistin and a second composition comprising E. coli strain Nissle 1917. Another particular kit further comprises an aminoglycoside, in particular neomycin. In this last embodiment, the aminoglycoside can be in the first composition, or in a third composition, separated from colistin.

In one aspect of the invention, the antibiotics are administered orally, but are specifically delivered and released in the ileum, the caecum or the colon, but not before the ileum. There are many means known for delivering a drug to the ileum, caecum or colon. Enteric coatings that protect the composition against gastric attack in the upper intestinal tract and release the active drug in the lower intestinal tract have been developed for years. See for example Singh et al., Modified-Release solid formulations for colonic delivery, in Recent patents on drug delivery & formulation, 2007, 1, 53-63; or Rubinstein et al., Colonic drug delivery; in Drug discovery today: technologies, 2005 Vol. 2, No. 1, 33-37. The delivery composition prevents any significant systemic absorption of the antibiotics thus avoiding or limiting any possibility of systemic toxic effect. It further prevents inactivation of the antibiotics during the intestinal transit. Release of the antibiotics in the ileum, caecum or colon can in particular be achieved by using the composition described in international applications WO2009/037264 and WO2010/103119, which are hereby incorporated by reference.

As mentioned above, in embodiments where more than one antibiotic is implemented, the antibiotics can be administered as two different products or combined in the same formulation at a given efficient dose. For example, the antibiotics can be combined in a single formulation for ileal/caecal/colonic delivery. In another illustrative embodiment, the antibacterials are formulated independently. In this embodiment, one may then include the two independent formulations in one single capsule or in distinct capsules (these distinct capsules can be associated in the same blister or separated in different blisters in the end-user product). In another illustrative embodiment, at least one, or all, of the antibiotics are formulated for delivery in the ileum, caecum or colon. In another embodiment, one antibiotic is formulated for ileal, caecal or colonic delivery, and other antibiotics are not formulated for delayed release. In this latter embodiment, both antibiotics can be included in the same product (for example in the same capsule, or tablet) or in different products (for example in different tablets or capsules, these different products being or not in the same blister).

The invention also relates to the above composition or kit for use in a method for providing elimination of Gram-negative resistant bacteria from the colon of a patient colonized by such bacteria, and the controlled repopulation of the commensal bacterial flora. In particular, the method prevents the dissemination of live resistant bacteria in the environment (for instance, but not only, following admission into a hospital) and prevents the occurrence of an infection caused by these bacteria in the colonized patient (for instance, but not only, before a surgical procedure) while allowing to repopulate the colon with bacteria known to be non-pathogenic and non-antibiotic resistant, thereby preventing colonization with harmful antibiotic-resistant and/or pathogenic bacteria.

The method of the invention can be implemented for eliminating Gram-negative resistant bacteria from the colon of a patient at risk before he develops an actual infection and then for controlling the repopulation of the colon thereby decontaminated.

The invention also relates to the above composition or kit, for use in a method of eliminating pathogenic microbes within the lumen of the intestinal tract, and minimizing the pathogenic alterations of the mucosa resulting from the action of compounds released by the infecting bacteria and then for controlling the repopulation of the colon thereby decontaminated.

The invention can also be used for eliminating Gram-negative bacteria from the colon of farm animals, in particular wherein the colonic bacteria to be targeted are Shiga toxin-producing Escherichia coli (or STEC), and then for controlling the repopulation of the colon thereby decontaminated.

The invention is also advantageous in that it can provide selective decontamination and controlled repopulation of the colonic bacterial flora in a patient to control outbreaks of antibacterial-resistant Gram-negative infections, such as nosocomial infections, in hospitals. Among others, said nosocomial infection may be caused by a) Gram-negative bacteria which are resistant to third generation cephalosporins by secretion of an extended spectrum beta-lactamase (ESBL) derived from the TEM or SHV beta-lactamase families, b) Gram-negative bacteria which are resistant to third generation cephalosporins by secretion of an extended spectrum beta-lactamase (ESBL) derived from CTX-M beta-lactamase family, or c) Gram-negative bacteria which are resistant to antibacterials by secretion of other types of enzymes such as carbapenemases of the KPC, VIM, OXA, NDM as well as other enzymatic families.

In a particular embodiment, the methods and kits of the present invention are used to suppress antibiotic-resistant bacteria, and in particular, multidrug resistant ESBL-producing bacteria from the colon and rebuild the commensal flora of carriers.

Efficiency of the method according to the invention can be monitored by measuring the counts of antibiotic-resistant bacteria in the faeces of the treated subject. For example, one can assay the decrease of the faecal ESBL-producing Gram-negative bacteria by well-known microbiological techniques. Monitoring can be done during a long period of time, for example at 5, 10, 20, 28 days after administration of the non-pathogenic, non-antibiotic-resistant E. coli strain (in particular of E. coli Nissle 1917 strain).

The present invention relates to a sequential treatment regimen composed of two phases: a ‘suppress’ and a ‘restore’ phases.

The ‘Suppress’ (or ‘decontamination’) phase, short and powerful, is preferably based on drug delivery systems such as those described in application WO2010/103119, for delivery to the ileum, caecum or colon of two Gram-negative antibacterial agents, in order to eradicate the carriage of antibiotic-resistant Gram-negative bacteria. The drug delivery systems used in application WO2010/103119 allow for the antibiotics comprised therein to be completely non-absorbable, as well as to prevent the side effects of those selected drugs at high doses in the upper gastrointestinal tract. The treatment is powerful in that the targeted concentrations of the two anti-microbial agents in the colon will be at least 6-8 times, preferably at least 10 times, over the Minimal Inhibitory Concentration (MIC) of the antibiotics for the bacterial strain(s) to be eliminated. The risk to observe the emergence of resistance is thus reduced. Assessment of the MIC of an antibiotic is well known to those skilled in the art.

The treatment is also short, since, for instance, the antibiotics can be administered during one to four days. The person skilled in the art will adapt the treatment regimen to the subject and its degree of infection by resistant bacteria. For example, the subject can be treated during one, two, three or four days, once or several times a day.

The ‘Restore’ phase (or recolonization phase), comprises a longer treatment step, from several days up to several weeks, where the patient flora is recolonized by a probiotic E. coli strain such as the E. coli Nissle 1917, in order to prevent the risk of a rebound effect with rapid recolonization by antibiotic-resistant Gram-negative bacteria. As mentioned above, this strain is well characterized, its genome has been sequenced, and it is not pathogenic due to the absence of known protein toxins. E. coli strain Nissle 1917 profoundly modulates the gut barrier to elevate the resistance of the gut to other microbial pathogens. Furthermore, this strain is well tolerated: among 3,807 patients studied, only 2.8% suspected side effects were reported, and none were considered a serious adverse event (Krammer et al., Z Gastroenterol. 2006 August; 44(8):651-656). The non-pathogenic, non-antibiotic resistant E. coli strain can start to be administered during or immediately after the suppress phase.

The suppress and restore phases can be implemented either in a hospital/clinic setting, or at home, depending on the condition of the subject to be treated.

### LEGENDS TO THE FIGURES

FIG. 1: synopsis of the study

FIG. 2: Comparisons (t-tests) between AUC of cefotaxime resistant Enterobacteriaceae counts (log₁₀) showed that in piglets treated with colistin 15 mg/kg, administration of E. coli Nissle 1917 (Gr 5) versus placebo (Gr 4) was associated with significantly lower counts of resistant Enterobacteriaceae (p=0.0045)

### EXAMPLE

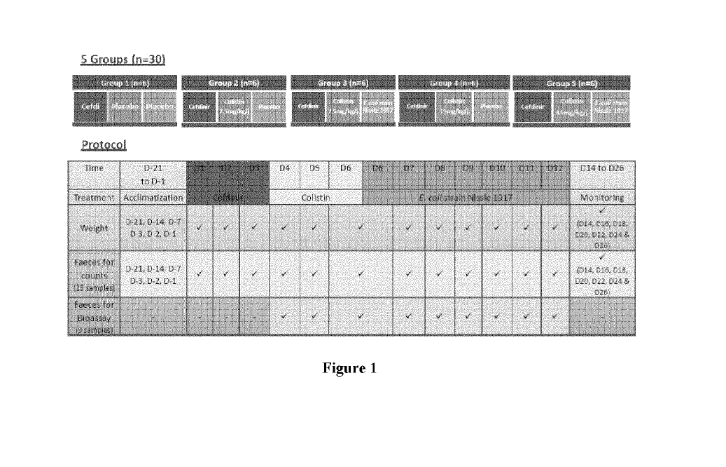

A piglet model was set up to demonstrate the sustained elimination of ESBL-producing Enterobacteriaceae (destroy and restore phases) in the gut flora. The model was used according to the synopsis depicted in FIG. 1.

### Synopsis of the Study

In summary, piglets were treated sequentially with:
cefdinir (4 mg/kg/d) during 2 days, in order to enhance the carriage of ESBL-producing bacteria,
colistin sulfate (destroy phase):

Colistin sulfate (Colivet), a non absorbable antibiotic, used in human decontamination, was given once per day orally in this experiment. Two regimens were tested: 7.5 mg/kg/d and 15 mg/kg/d, equivalent to human doses 500 mg/d and 1000 mg/d, respectively. The duration of the treatment was 3 days. The objective of such very high doses, given over a short period of time, was to obtain a rapid eradication of ESBL-producing Enterobacteriaceae without the emergence of strains resistant to colistin.
E. coli Nissle strain 1917 (EcN1917, Mutaflor). The dose was calculated to administrate the highest dose recommended in humans: 2 enteric-coated capsules of 2.5-25×10⁹ viable bacteria, given orally, once daily during 6 days.

### Methods

### Microbiological Assay of Colistin:

Fecal concentrations of colistin were measured by a microbiological assay using agar medium 10 (Difco, Becton-Dickinson, Le Pont-de-Claix) and Bordetella bronchiseptica ATCC4617 as indicator strain. The limit of detection was 1.5 μg of colistin base per g of feces.

Fresh faecal samples were collected from each piglet, diluted ten-fold (w/v) in peptone broth containing 30% glycerol, and faecal suspension aliquots were stored at −80° C. until microbiological analysis.

### Bacterial Counts in Feces

Before analysis, frozen aliquots were thawed by standing approximately 1 hr at room temperature and faecal suspensions were serially diluted in saline before plating on appropriate agar plates.

Total Enterobacteriaceae were counted using Drigalski agar (BioRad Laboratories, Marnes-La-Coquette, France) plates.

Cefotaxime- and colistin-resistant Enterobacteriaceae were counted using Drigalski agar supplemented with 1.5 mg/L cefotaxime and 10 mg/L colistin base respectively.

Bacterial counts which were below the detection threshold (2 log CFU/g of faeces) were considered to be equal to the 2 log CFU/g for statistical analysis.

### Results

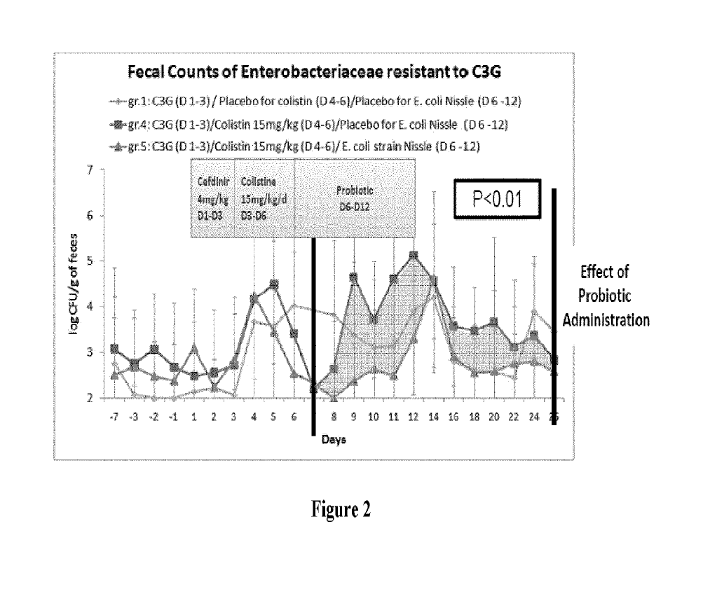

The results showed that colistin administered for three days followed by six days of treatment using the probiotic E. coli strain Nissle 1917 led to a rapid and efficacious decolonization of ESBL-producing enterobacteria, followed by a sustained recolonisation by non-ESBL strains (see FIG. 2).

Importantly, no selection of strains resistant to colistin was observed during follow-up (14 days). Thus, with the method of the present invention, very high concentrations of antibiotics can be obtained in the colon. The risk of emergence of resistance is reduced by a sustained recolonisation with non-ESBL producer strains.

In the destroy phase, the best efficacy to eradicate ESBL-producing enterobacteria was obtained with 15 mg/kg/d colistin sulfate: the faecal concentrations were at least 10-fold higher than the MIC.

In the restore phase, the best efficacy was obtained in the group which had received the high dose of colistin.

1. A method for the repopulation of the commensal flora of the colon of a subject in need thereof who has been previously subjected to a Gram-negative bacteria selective decontamination of its colon, comprising administering to the subject a non-pathogenic, non-antibiotic-resistant Escherichia coli strain.

2. The method according to claim 1, wherein the non-pathogenic, non-antibiotic-resistant E. coli strain is E. coli strain Nissle 1917.

3. The method according to claim 1, wherein the decontamination has been previously carried out by administering at least one anti-Gram-negative antibiotic.

4. A method for the elimination of antibiotic-resistant Gram-negative bacteria in the colon of a subject in need thereof comprising:
a) a step of elimination of Gram-negative bacteria in the colon;
b) a step of repopulating the colon of the subject with a non-pathogenic, non-antibiotic-resistant E. coli strain.

5. The method according to claim 4 wherein the non-pathogenic, non-antibiotic-resistant E. coli strain is E. coli strain Nissle 1917 (DSM 6601 strain).

6. The method according to claim 4, wherein step a) comprises administration of at least one antibiotic effective against Gram-negative antibiotic-resistant bacteria present in the colon.

7. The method according to claim 6, wherein one or more of the at least one antibiotic is formulated such as to be delivered to a part of the intestine of the subject selected from the ileum, the caecum or the colon, but not before the ileum.

8. The method according to claim 4, wherein step a) comprises administration of a peptide antibiotic.

9. The method according to claim 6, comprising administration of an aminoglycoside to the subject.

10. The method according to claim 9, wherein the aminoglycoside is neomycin.

11. The method according to claim 10, wherein the aminoglycoside is formulated such as to be delivered to a part of the intestine of the subject selected from the ileum, the caecum or the colon, but not before the ileum.

12. The method according to claim 4, step a) comprising the administration of colistin and neomycin to the subject.

13. The method according to claim 12, wherein colistin is formulated such as to be delivered to a part of the intestine of the subject selected from the ileum, the caecum or the colon, but not upstream from the ileum.

14. The method according to claim 12, wherein neomycin is formulated such as to be delivered to a part of the intestine of the subject selected from the ileum, the caecum or the colon, but not upstream from the ileum.

15. (canceled)

16. The method according to claim 8, wherein the peptide antibiotic is colistin.

17. The method according to claim 9, wherein the aminoglycoside is selected from the group consisting of neomycin, amikacin, arbekacin, gentamicin, kanamycin, netilmicin, paromomycin, rhodostreptomycin, streptomycin, tobramycin, spectinomycin and apramycin.

18. The method according to claim 13, wherein neomycin is formulated such as to be delivered to a part of the intestine of the subject selected from the ileum, the caecum or the colon, but not upstream from the ileum.
